(19) United States
(12) Patent Application Publication
(10) Pub. No.: US 2008/0046059 A1
(43) Pub. Date: Feb. 21, 2008

(54) LEAD INCLUDING A HEAT FUSED OR FORMED LEAD BODY

(76) Inventors: Paul E. Zarembo, Vadnais Heights, MN (US); Mohan Krishnan, Shoreview, MN (US); David Durand, St. Paul, MN (US); Donna Osterkamp, Forest Lake, MN (US); Daniel J. Cooke, Roseville, MN (US)

Correspondence Address: SCHWEGMAN, LUNDBERG & WOESSNER, P.A., P.O. BOX 2938, MINNEAPOLIS, MN, 55402, US

(21) Appl. No.: 11/462,479

(22) Filed: Aug. 4, 2006

(51) Int. Cl. A61N 1/00

(52) U.S. Cl. 607/122

(57) ABSTRACT

An implantable lead comprises a lead body extending from a lead proximal end portion to a lead distal end portion. In one example, the lead body may comprise a heat-formed bias portion. In another example, an outer insulator is fused to the lead body. In such an example, a lead body fusable plug may be disposed distal to at least one conductor. In another example, the lead comprises an inner boot and an outer boot fused to one another. In another example, the lead includes an atraumatic tip fused to the lead distal end portion. In another example, the lead body is reducable in size using heat shrink tubing. In yet another example, two or more lead sections may be interconnected using an outer insulator fused to the respective lead bodies. In a further example, a stiffener member is fused to the lead body adjacent a lead component.

### TECHNICAL FIELD

This patent document pertains generally to leads for linking medical devices with selected bodily tissue to be sensed or stimulated by such devices. More particularly, but not by way of limitation, this patent document pertains to a lead including a heat fused or formed lead body.

### BACKGROUND

Leads represent the electrical link between an implantable medical device (referred to as “IMD”), such as a pacer or defibrillator, and a subject's cardiac or other bodily tissue, which is to be sensed or stimulated. A lead generally includes a lead body that contains one or more electrical conductors extending from a proximal end portion of the lead to an intermediate or distal end portion of the lead. The lead body includes insulating material for covering and electrically insulating the electrical conductors. The proximal end of the lead further includes an electrical connector assembly couplable with the IMD, while the intermediate or distal end portions of the lead includes one or more electrodes that may be placed within, on, or near a desired sensing or stimulation site within the body of the subject.

Some subjects require a lead system having the ability to sense or stimulate at multiple locations within, on, or near their heart or heart vessels. In the past, a common practice for a subject requiring multi-site sensing or stimulation was to provide two or more separate leads disposed at different cardiac locations. One lead would be implanted at a first site, while at least another lead would be implanted at a second site, spaced from the first site. Drawbacks of having two or more separate leads can be numerous. As one example, the complexity and time required to implant two or more leads may be much greater than what is required for implanting one lead. In addition, the two or more leads may mechanically interact with one another after implantation resulting in dislodgement of one or more leads. Another problem is that as more leads are implanted within, on, or near the heart or heart vessels, the ability to add further leads is reduced.

Implantable leads, such as those used for cardiac sensing or stimulation, should have the ability to remain fully assembled and leak resistant despite constant flexing or bending, which may be encountered by the implanted leads with each ventricular or atrial contraction of cardiac tissue or forces applied to the leads during implantation, repositioning, or extraction. In addition, implantable leads should be designed to resist failure due to extended contact with in vivo bodily fluids, such as blood.

Recently, there has been a high level of interest in designing leads having lead bodies with a reduced size (i.e., lead body diameter). A reduced diameter lead, among other things, advantageously limits the negative surgical effects of lead implantation. In addition, a smaller lead size can advantageously provide access to certain (hard to reach) tissues and structures without compromising blood flow.

What is needed is a lead having a small lead body size, which still possesses the ability to sense or stimulate at multiple cardiac locations. What is further needed is a lead that is manufacturable in a relatively quick, efficient, and cost effective manner. Further yet, what is needed is a reliable lead that is easy to implant within, and extract out of, a subject.

### SUMMARY

A lead comprises a lead body extending from a lead proximal end portion to a lead distal end portion, with a lead intermediate portion therebetween. At least one tissue sensing/stimulation electrode is disposed along the lead body, and is connected to one or more terminal conductors at the lead proximal end portion. The lead body includes at least one heat-formed bias portion at the lead intermediate or distal end portions. In one example, the bias portion includes at least one of a cylindrical, oval, or cam-like helical shape.

Another lead comprises a lead body having one or more longitudinally extending lumens therein. A first conductor is received in, and extends along, a first lumen. A thermoplastic outer insulator, such as an insulator comprising polyurethane, is disposed around a portion of the lead body and fused thereto.

Another lead comprises a lead body housing a coil conductor and at least one cable conductor, or alternatively, a plurality of cable conductors and no coil conductor. In one example, the coil conductor is surrounded by a polymer coating, such as polytetrafluoroethylene. In another example, the at least one cable conductor is surrounded by a polymer coating, such as ethylene tetrafluoroethylene. An outer insulator surrounds a portion of the lead body. A length of heat shrink tubing is disposed around the outer insulator, such that when the tubing is heated, the outer insulator and the lead body are diametrically compressed. In such an example, the outer insulator becomes fused with portions of the lead body, after which the heat shrink may be removed.

Yet another lead comprises a lead body adapted to carry signals, the lead body extending from a lead proximal end portion to a lead distal end portion, and having a lead intermediate portion therebetween. A connector assembly is located at the lead proximal end portion. A lead terminal boot including an inner boot and an outer boot is disposed distally to the connector assembly. The inner boot is fusable with the lead body on an inner surface and fusable with the outer boot on an outer surface. In one example, both the inner and outer boots comprise polyurethane or silicone rubber. A further lead comprises a lead body having a flexible tip portion. The flexible tip portion being optionally more flexible than the lead body and fused to the lead body at one or more fusion zones.

A further lead comprises a lead body having one or more longitudinally extending lumens. At least one conductor is received in, and extends along, the one or more lumens. A lead component is disposed on the lead body and is abutted on each side by an outer insulator surrounding a portion of the lead body. A stiffener member is disposed between the lead body and the outer insulator and is fused to portions thereof In varying examples, the stiffener member comprises a thermoplastic tubular structure having a stiffer modulus of elasticity than a modulus of elasticity of the lead body and the outer insulator.

A lead assembly comprising a proximal lead section and a distal lead section is also discussed. An end portion of the proximal lead section is disposed adjacent to an end portion of the distal lead section. An outer insulator is disposed around an outer surface of the proximal lead section end portion and the distal lead section end portion. A length of heat shrink tubing is disposed around the outer insulator, such that a substantial length of the outer insulator is covered. The heat shrink tubing diametrically compresses the outer insulator, a lead body of the proximal lead section, and a lead body of the distal lead section when heated. In such an example, the outer insulator becomes fused with the proximal and distal lead sections lead bodies, after which the heat shrink tubing is removed.

The leads described herein provide numerous advantages over conventional lead designs including a small-sized lead body (e.g., sub 5-French, such as about 4-French), which advantageously provides for easier and deeper lead delivery and may provide for lower sensing/stimulation thresholds. In one such example, the present leads provide a small-sized lead with multiple (e.g., three or more) conductors and corresponding tissue sensing/stimulation electrodes. Multiple conductors and electrodes allow for electrode switching to occur, which in turn prevents extra (unnecessary) bodily tissue stimulation and optimizes a variety of other sensing/stimulation related parameters (e.g., parameters relating to the selection of electrodes/vectors with desirable thresholds for longer device life, maintaining capture should micro-lead dislodgement occur, or optimizing hemodynamics), as further described in Hansen, et al., U.S. Patent Application titled “MULTI-SITE LEAD/SYSTEM USING A MULTI-POLE CONNECTION AND METHODS THEREFOR,” Ser. No. 11/230,989, filed Sep. 20, 2005, which is hereby incorporated by reference in its entirety.

Several other advantages are also made possible by the present leads. In some examples, the leads reduce or eliminate the reliance on adhesives for lead manufacture. Advantageously, by reducing or eliminating reliance on adhesives, manufacturing efficiency can be increased (e.g., a manufacturer may not need to wait for adhesives to cure), and lead joint failure caused by adhesive bond strength decreasing over time (e.g., due to moisture, body heat, reactions with bodily fluids or improper adhesive or surface preparation) can be reduced or eliminated. These and other examples, features, and advantages of the present leads will be set forth in part in the detailed description, which follows, and in part will become apparent to those skilled in the art by reference to the following description and drawings, or by practice of the same.

### BRIEF DESCRIPTION OF THE DRAWINGS

In the drawings, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is a schematic view illustrating an implantable lead system and an environment in which the lead system may be used, as constructed in accordance with at least one embodiment.

FIG. 2A is a schematic view illustrating an implantable lead system for delivering or receiving signals to or from a heart, as positioned and constructed in accordance with at least one embodiment.

FIG. 2B is a schematic view illustrating an implantable lead system for delivering or receiving signals to or from a heart, as positioned and constructed in accordance with at least one embodiment.

FIG. 3 is a plan view of an implantable lead, as constructed in accordance with at least one embodiment.

FIG. 4A is a schematic view of portions of an implantable lead and a lead manufacturing apparatus, as constructed in accordance with at least one embodiment.

FIG. 4B is a schematic view of a portion of an implantable lead and a lead manufacturing apparatus, as constructed in accordance with at least one embodiment.

FIG. 4C is a schematic view illustrating implantable leads and an environment in which the leads may be used, as constructed in accordance with at least one embodiment.

FIG. 4D is an isometric view of a lead manufacturing apparatus, as constructed in accordance with at least one embodiment.

FIG. 5 is a cross-sectional view of an implantable lead taken along line 5-5 of FIG. 3, as constructed in accordance with at least one embodiment.

FIG. 6 is a cross-sectional view of an implantable lead taken along line 6-6 of FIG. 3, as constructed in accordance with at least one embodiment.

FIG. 7 is a lengthwise cross-sectional view illustrating a portion of an implantable lead, as constructed in accordance with at least one embodiment.

FIG. 8A is a lengthwise cross-sectional view illustrating portions of an implantable lead, as constructed in accordance with at least one embodiment.

FIG. 8B is a lengthwise cross-sectional view illustrating portions of an implantable lead, as constructed in accordance with at least one embodiment.

FIG. 9 is a lengthwise cross-sectional view illustrating a distal portion of an implantable lead, as constructed in accordance with at least one embodiment.

FIG. 10 is a lengthwise partial cutaway view illustrating a portion of an implantable lead, as constructed in accordance with at least one embodiment.

FIG. 11A is a lengthwise cross-sectional view illustrating an interconnection between a proximal lead section and a distal lead section, as constructed in accordance with at least one embodiment.

FIG. 11B is a lengthwise exterior view illustrating the interconnection of FIG. 11A, as constructed in accordance with at least one embodiment.

FIG. 12 is a lengthwise cross-sectional view illustrating a lead component portion of an implantable lead, as constructed in accordance with at least one embodiment.

### DETAILED DESCRIPTION

The following detailed description includes references to the accompanying drawings, which form a part of the detailed description. The drawings show, by way of illustration, specific embodiments in which the present leads may be practiced. These embodiments, which are also referred to herein as “examples,” are described in enough detail to enable those skilled in the art to practice the present leads. The embodiments may be combined, other embodiments may be utilized or structural and logical changes may be made without departing from the scope of the present leads. It is also to be understood that the various embodiments of the present leads, although different, are not necessarily mutually exclusive. For example, a particular feature, structure or characteristic described in one embodiment may be included within other embodiments. The following detailed description is, therefore, not to be taken in a limiting sense, and the scope of the present leads are defined by the appended claims and their legal equivalents.

In this document the terms “a” or “an” are used to include one or more than one; the term “or” is used to refer to a nonexclusive or, unless otherwise indicated; and the term “subject” is used synonymously with the term “patient.” In addition, it is to be understood that the phraseology or terminology employed herein, and not otherwise defined, is for the purpose of description only and not of limitation.

The leads discussed herein advantageously provide, among other things, one or more of the following: a small-sized lead body; an ability to sense or stimulate at multiple cardiac tissue locations; an improved reliability (over conventional leads) in an in vivo environment; easy lead implantation and extraction; left-ventricular positioning; or varying stiffness along the lead body. The following text and associated figures begin with a generalized discussion of a lead system (including one or more leads and a medical device), and an environment in which the lead system may be used. The text and figures continue with a more detailed discussion of the present leads, and various characteristics that such leads may comprise in order to provide one or more of the aforementioned advantages. Although the following discusses many lead characteristics individually or in specific combinations, any combination of the lead characteristics described herein is within the scope of the present subject matter.

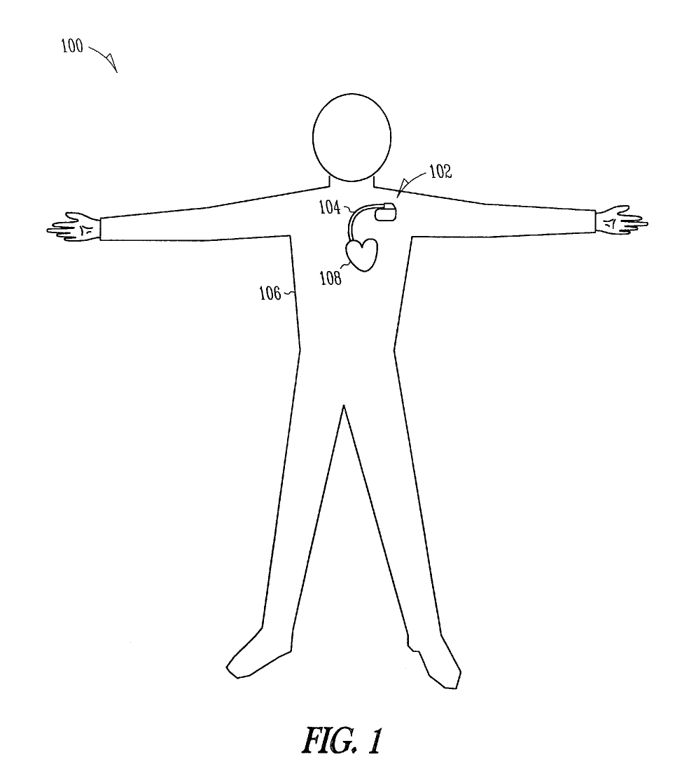

Turning now to the drawings, and initially to FIG. 1, which illustrates a lead system 100 and an environment 106 (e.g., a subcutaneous pocket made in the wall of a subject's chest, abdomen, or elsewhere) in which the lead system 100 may be used. In varying examples, the lead system 100 may be used for delivering or receiving electrical pulses or signals to stimulate or sense a heart 108 of a subject 106. As shown in FIG. 1, the lead system 100 includes an IMD 102 and at least one implantable lead 104. The IMD 102 generically represents, but is not limited to, cardiac function management (referred to as “CFM”) systems such as pacers, cardioverters/defibrillators, pacers/defibrillators, biventricular or other multi-site resynchronization or coordination devices such as cardiac resynchronization therapy (referred to as “CRT”) devices, sensing instruments, or drug delivery systems.

Among other things, the IMD 102 includes a source of power as well as an electronic circuitry portion. In one example, the electronic circuitry includes microprocessors to provide processing, evaluation, or to determine and deliver electrical shocks or pulses of different energy levels and timing for ventricular defibrillation, cardioversion, or pacing of the heart 108, such as in response to sensed cardiac arrhythmia including fibrillation, tachycardia, or bradycardia. In another example, the IMD 102 is a battery-powered device that senses intrinsic signals of the heart 108 and generates a series of timed electrical discharges.

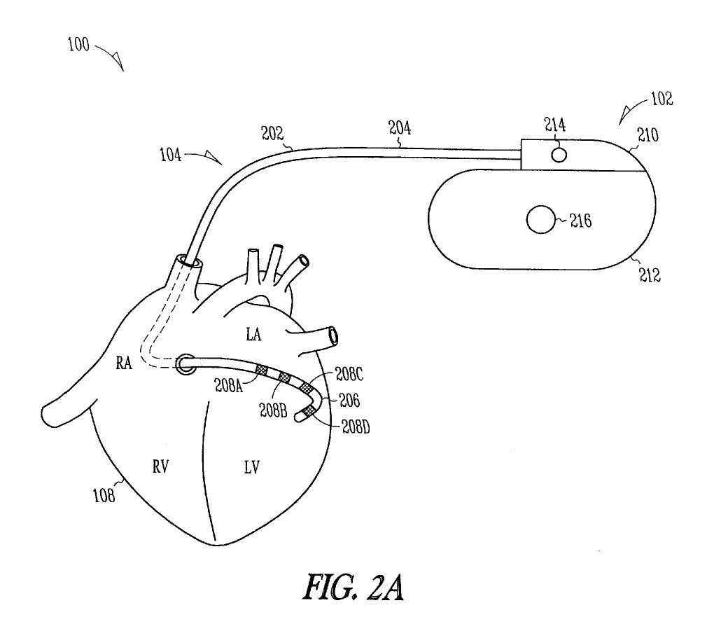

FIGS. 2A-2B are schematic views of a lead system 100 including an IMD 102 and at least one implantable lead 104. As shown, the lead 104 includes a lead body 202 extending from a lead proximal end portion 204, where it is couplable with the IMD 102, to a lead distal end portion 206, which is positionable within, on, or near a heart 108 or heart vessels when fully implanted. In this example, the lead distal end portion 206 includes at least one electrode, such as four electrodes 208A, 208B, 208C, 208D, that electrically link the lead 104 with the heart 108. At least one conductor coil 502 or cable 504 (see, e.g., FIG. 5), electrically couples the electrodes 208A, 208B, 208C, 208D with the lead proximal end portion 204 and thus, the electronic circuitry of the IMD 102. The conductors 502, 504 carry electrical current in the form of pulses or shocks between the IMD 102 and the electrodes 208A, 208B, 208C, 208D. The lead 104 may be installed using either over-the-wire (referred to as “OTW”) or non-OTW techniques, such as stylet driving or catheter delivering.

In the examples shown in FIGS. 2A-2B, the lead 104 is a multi-electrode lead including a proximal electrode 208A, two intermediate electrodes 208B, 208C, and a distal electrode 208D. Each of the electrodes 208A, 208B, 208C, 208D may, for example, comprise ring electrodes or single or multi-filar shock coil electrodes and are independently connected to a separate (corresponding) electrically conductive terminal within a header 210 of the IMD 102. The header 210 is affixed to a hermetically sealed housing 212, which may be formed from a conductive metal such as titanium, and which carries the electronic circuitry of the IMD 102. In this example, the header 210 includes a header electrode 214 and the housing 212 includes a housing electrode 216, both of which may be used in one or more electrode configurations for sensing or stimulating heart 108, as further described in Hansen, et al., U.S. Patent Application titled “MULTI-SITE LEAD/SYSTEM USING A MULTI-POLE CONNECTION AND METHODS THEREFOR,” Ser. No. 11/230,989, filed Sep. 20, 2005.

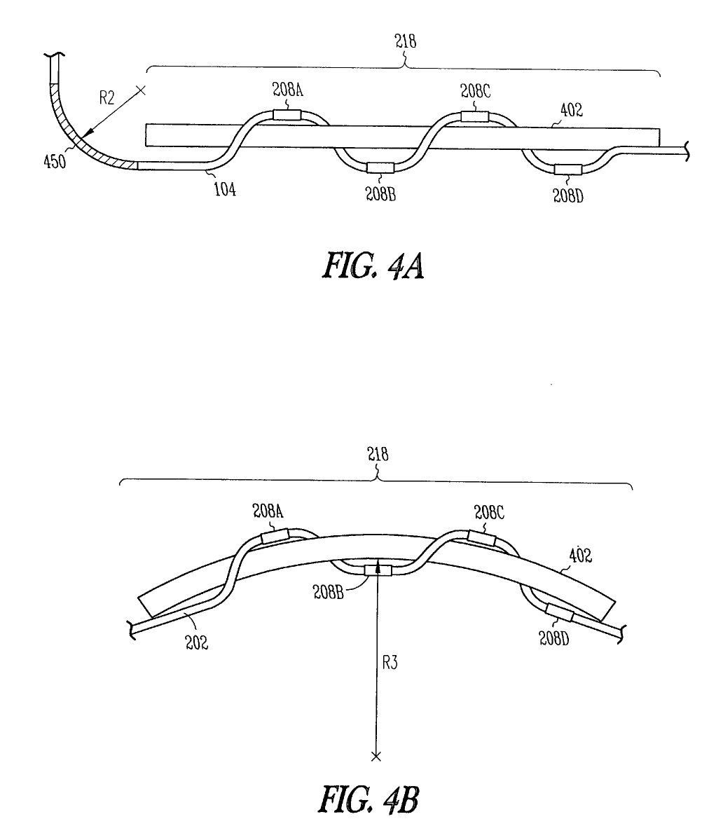

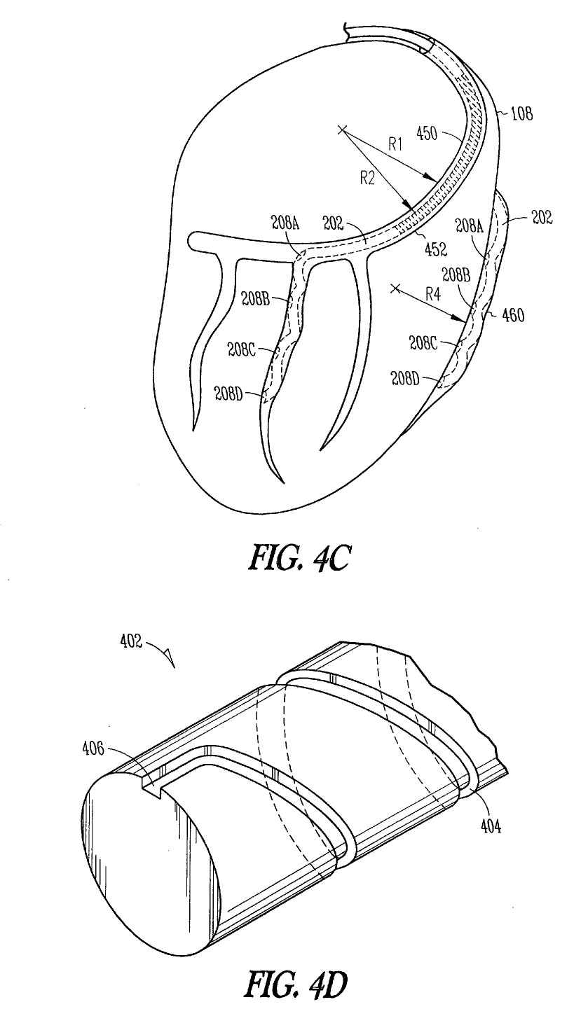

FIGS. 2A-2B each illustrate a lead distal portion 206 disposed in a left ventricle (referred to as “LV”) of the heart 108. Such exemplary dispositions of the lead 104, specifically the lead distal portions 206, are useful for sensing or delivering stimulation energy to a left side of the heart 108 for treatment of heart failure or other cardiac disorders requiring therapy be delivered to the heart's left side. FIG. 2B further illustrates that the lead body 202 may include at least one heat-formed bias portion 218 to urge the one or more electrodes 208A, 208B, 208C, 208D disposed thereon against a vessel wall (or other portion of heart 108) for pacing or sensing of the same or to stabilize a position of lead distal end portion 206 within the cardiac vessel. As discussed below in association with FIGS. 4A-4B, the heat-formed bias portion 218 may be formed using, in part, heat from a heat source in combination with a cylindrical or other appropriately shaped mandrel 402. Although not shown in FIGS. 2A-2B, other dispositions of the lead intermediate or distal end portions 206 within, on, or about the heart 108 are also possible without departing from the scope of the present subject matter.

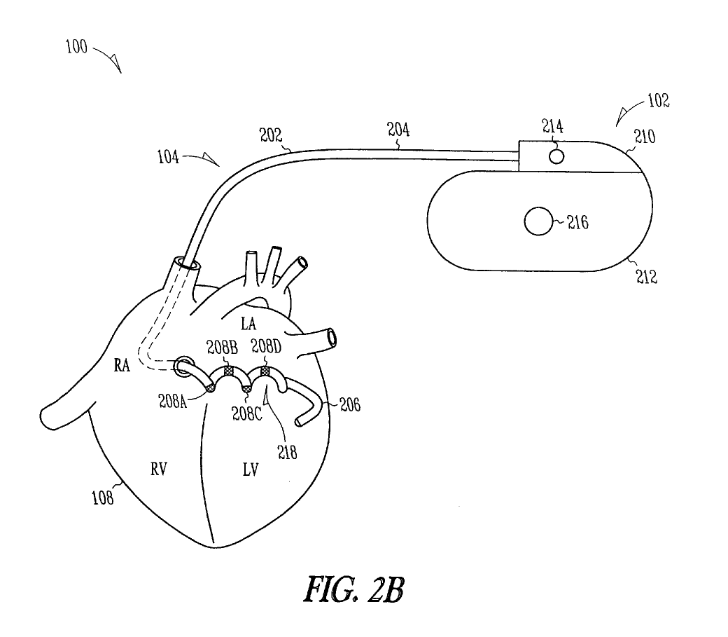

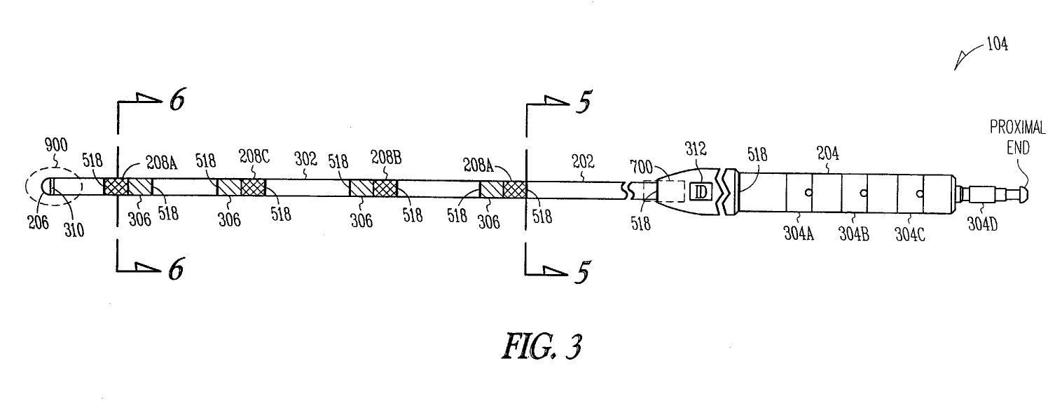

FIG. 3 illustrates a plan view of an implantable lead 104. As shown, the lead 104 includes a lead body 202 extending from a lead proximal end portion 204 to a lead distal end portion 206 and having an intermediate portion 302 therebetween. In one example, the lead body 202 comprises biocompatible tubing such as medical grade polyurethane. In another example, the lead body 202 comprises medical grade silicone rubber or silicone rubber coated by polyurethane. As discussed above in association with FIG. 1, a lead system 100 includes, among other things, at least one lead 104 for electrically coupling an IMD 102 (FIG. 1) to bodily tissue, such as a heart 108 (FIG. 1), which is to be sensed or stimulated by one or more electrodes, such as four electrodes 208A, 208B, 208C, 208D. It should also be understood that the lead 104 may also include means for sensing other physiological parameters, such as pressure, acceleration, sound, oxygen saturation, temperature, or the like. As one example, in addition to electrodes 208A, 208B, 208C, 208D, the lead 104 may include one or more drug collars 306, such as a steroid collar. For sealing of the lead 104, retainment of the electrodes 208 or drug collars 306, or other lead manufacturing reasons, the lead body 202 may be fused 518 proximally and distally to the electrodes 208 and drug collars 306, respectively.

As shown in FIG. 3, the lead proximal end portion 204 includes four terminal connections 304A, 304B, 304C, 304D disposed therealong. The electrodes 208A, 208B, 208C, 208D are each adapted to sense or stimulate the heart 108 (FIG. 1) and are electrically coupled to the terminal connections 304A, 304B, 304C, 304D via at least one coil or cable conductor 502, 504 (FIG. 5) contained within the lead body 202, such as in one or more longitudinally extending lumens 506, 508, 510, 512 (FIG. 5). The lead proximal end portion 204 and the terminal connections 304A, 304B, 304C, 304D disposed therealong are sized and shaped to couple to a multi-pole connector cavity, which may be incorporated into a header 210 (FIGS. 2A-2B) of the IMD 102. It is through the coupling between the lead proximal end portion 204 and the multi-pole connector cavity that electrodes 208A, 208B, 208C, 208D are electrically coupled to electronic circuitry of the IMD 102. Although FIG. 3 illustrates a lead 104 having four terminal connections 304 and four electrodes 208, the present subject matter is not so limited. In other examples, the lead 104 comprises more than or less than four terminal connections 304 and electrodes 208.

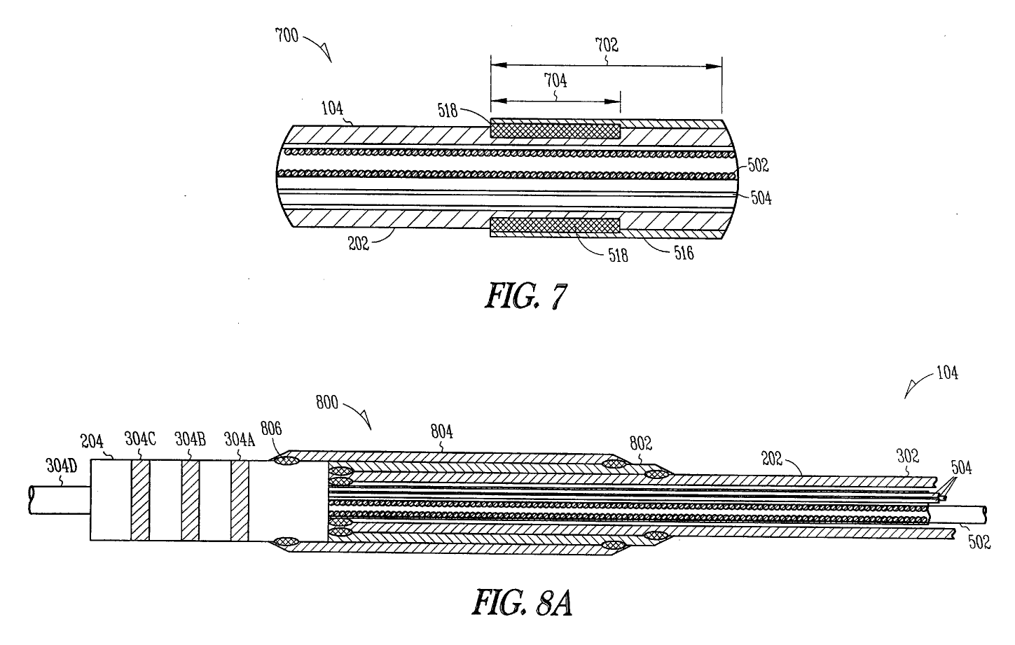

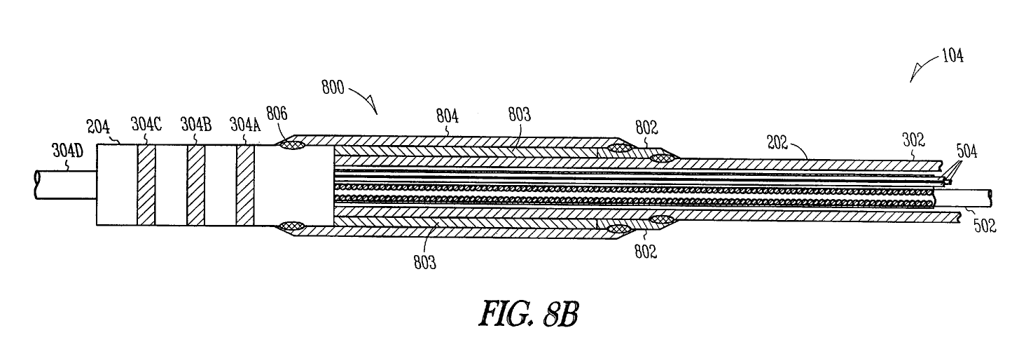

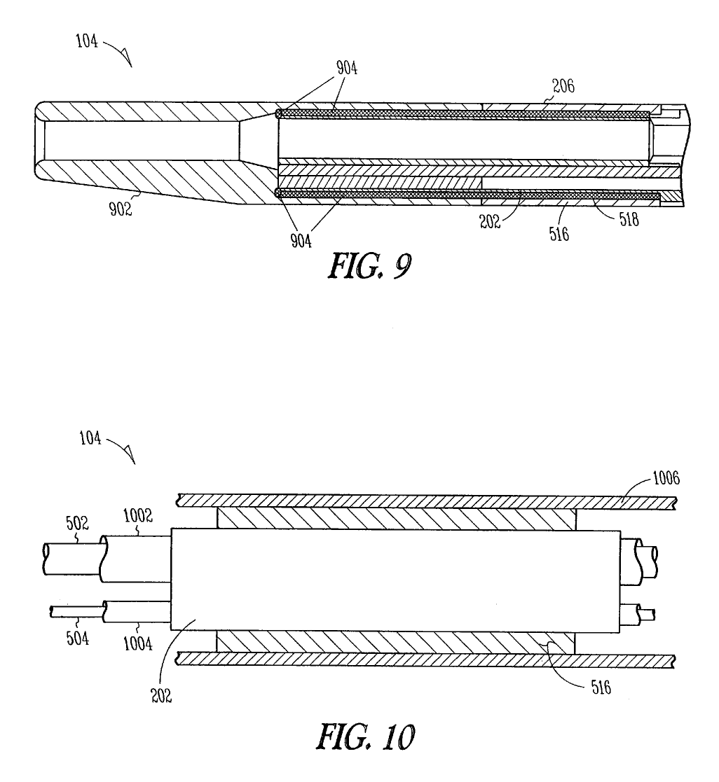

Optionally, the lead distal end portion 206 may include a fluoromarker 310 fused therewithin. In one such example, the fluoromarker 310 comprises polyurethane filled with barium sulfate. As another option, a portion of the lead 104, such as the lead proximal end portion 204, may include an identification label 312 fused therewithin. In one such example, the identification label 312 comprises (white) titanium oxide polyurethane. The lead portions enclosed by the phantom lines 700 and 900 in FIG. 3 are illustrated in more detail in FIGS. 7 and 9, respectively. As yet another option, a fixation member may be disposed on, and fused to, the lead body 202.

FIG. 4A is a schematic view illustrating portions of an implantable lead 104 having a lead body 202 and a lead manufacturing apparatus, such as a mandrel 402. As shown, but as may vary, the lead 104 includes four electrodes 208A, 208B, 208C, 208D. A portion 218 of the lead 104 comprises a heat-formed 2-D or 3-D bias, which facilitates electrode placement and contact (with the heart 108 (FIG. 1) or vessels associated therewith), or fixation of the lead within, on, or near the same. The shape of lead body 202 allows for spatial orientation of the electrodes 208A, 208B, 208C, 208D. Depending, in part, on a shape of the heat-formed bias portion 218, the electrodes may be arranged at 90 degrees form each other, placed on one side of the bias, or progressively spaced, for example. The lead body 202 may comprises an environment adaptable polymer material chosen to adapt (e.g., creep) to its coronary or other surroundings over time. For instance, the polymer material may be chosen based on its glass transition temperature (T_{g}). It is believed that over time the heat-formed 2-D or 3-D bias of the lead may adapt to a geometry of the coronary vasculature in which it is implanted, thereby establishing greater fixation.

The heat-formed bias portion 218 may assume various configurations. According to one lead forming method, the lead body 202 is wrapped around a cylindrical or other desired shaped (e.g., oval, cam-shaped, J-shaped, or sinusoidal) mandrel 402 in a helical or other manner and heated (e.g., using a moving heated die, laser such as CO₂, infra-red, etc.). In addition to its cross-sectional shape, the mandrel 402 may also include a non-linear longitudinal shape, such as a curve 470 (FIG. 4B) having a radius R₃, which it imparts to the lead body 202 wrapped therearound. The radius R₃ may allow for the lead body 202 to closely match a geometry R₄ of a portion of the heart 108, such as the geometry of a coronary branch vein 460 as shown in FIG. 4C. The heat, in combination with the mandrel 402, may result in the lead body 202 including a helical biased portion. In varying examples, the heat-formed bias portion 218 has (in a relaxed state) a lateral extension larger than a diameter of the lead body 202, and an elasticity that is substantially comparable to that of the heart portion where implantation is expected, thereby encouraging intimate contact between the same. In another example, the lead body 202 is formed to include an oval or trilobular helical heat-formed bias, which my provide a desired electrode orientation or increased retention force. In yet another example, the lead body 202 is formed to include a shape resembling a sinusoidal curve.

The heat-formed bias portion 218 may provide many advantages to the present lead 104 over conventional leads. As one example, the biased portion 218 allows for the creation of a small-sized lead body 202, which still adequately maintains a desired position within the desired cardiac or other region as the bias portion provides position retention to the lead. Accordingly, other lead fixation devices, such as tines, corkscrews, etc. may not needed, and therefore need not be incorporated into such a lead. This, in turn, may aid in further reducing the size of the lead body 202. As another example, the biased portion 218 helps to stabilize positions of the one or more electrodes 208A, 208B, 208C, 208D for long periods of time and may result in lower sensing or stimulation thresholds due to intimate contact between the electrodes and portions of the heart 108, such as the vessels associated therewith. As yet another example, the biased portion 218 may advantageously orient the lead electrodes 208 in a coronary vein, for instance, with respect to a heart 108 wall. For implantation or extraction of lead 104, a physician may use an introductory catheter, a stylet, or a guidewire to straighten the heat-formed bias portion 218 of the lead body 202. When the lead 104 is positioned as desired, the introductory catheter, stylet, or guidewire can be withdrawn so that the biased portion 218 assumes its biased configuration.

As further shown in FIG. 4A, the implantable lead 104 may optionally include a curve portion 450 proximal or distal to the bias portion 218. The curve portion 450 may include a relatively stiff curve configured to orient and fix portions of the lead body 202 against the heart 108, as shown in FIG. 4C. In one example, the curve portion 450 may be formed by heat or by thicker or stiffer polymers (e.g., polyurethane (referred to as “PU”)) fused to the lead body 202. In FIG. 4C, a great cardiac vein 452 of the heart 108 is shown to have a radius R₁, which is substantially similar with the radius R₂ of the curve portion 450. As illustrated in FIG. 4B, the cylindrical mandrel 402 may include a groove 404 helically positioned therearound, which provides a track for the lead 104 to follow during manufacture and thereby may impart characteristics such as pitch, spacing, and diameter to the heat-formed bias portion 218. In this example, a distal portion of the mandrel 402 includes a transition and exit region 406 in which the lead 104 may be transitioned from a helical shape to a substantially straight shape.

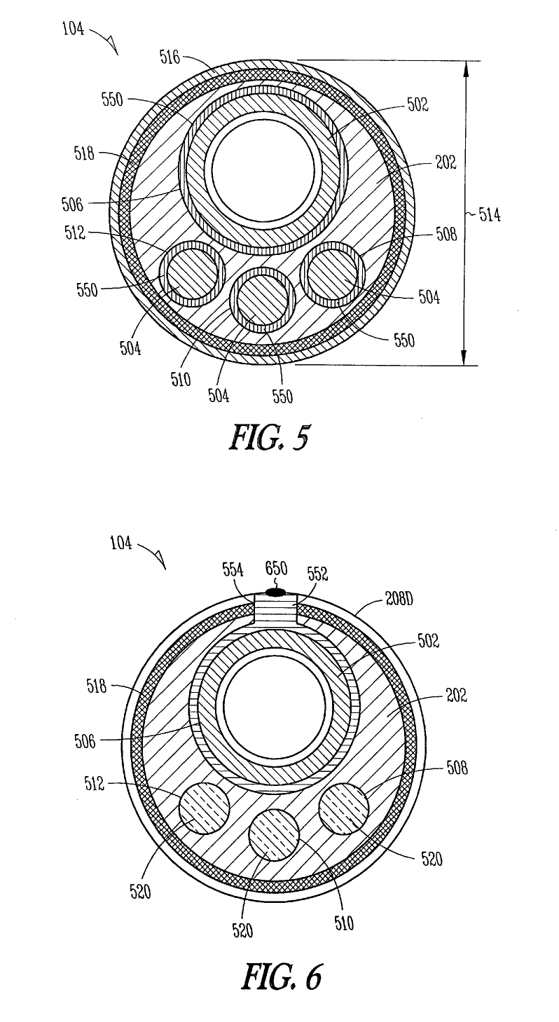

FIGS. 5-6 illustrate two exemplary cross-sectional configurations of a lead body 202. As shown in these examples, one or more lead components (e.g., comprising PU, ethylene tetrafluoroethylene (referred to as “ETFE”), polytetrafluoroethylene (referred to as “PTFE”), such as expanded PTFE, or other thermoplastics) may be fused together to bind such components to one another. Advantageously, fusion of one or more lead components allows for a small-sized lead body 202 to be created, as the need for binding adhesives (and its accompanying size) is reduced or eliminated. The reduction in lead body diameter may provide room for, among other things, a steroid drug collar 306 (FIG. 3) or deeper cardiac implantation of the lead. In addition, the fusion of lead components may provide for increased lumen sealing ability (e.g., around the electrodes) or lead body 202 (axial or torsional) strength.

The cross-sectional views of FIGS. 5-6 illustrate that the present lead body 202 may include one or more lumens, such as one coil lumen 506 and three cable lumens 508, 510, 512. As shown in each FIG., but as may vary, a coil conductor 502 is disposed within lumen 506 and cable conductors 504 are disposed within each of lumens 508, 510, 512. In one example, such a quad-lumen lead has an outer diameter 514 of about 4-French (0.053″). In particular, the cross-section of FIG. 5 illustrates one example of a lead 104 at a location proximal to a first electrode 208A. At this lead location, the coil conductor 502 and the cable conductors 504 each comprise insulative tubing 550, such as ETFE tubing, around an outer surface thereof Optionally, as shown in FIG. 5, an outer insulator 516 may be fused 518 to the inner multi-lumen lead body 202, thereby providing sealing or redundant insulation to the lead 104. In addition to sealing or insulating, the outer insulator 516 may be used to hold lead components (e.g., an electrode, lead terminal boot, drug collar, suture sleeve, or label) in place or provide a blend to the lead body's 202 outside surface.

The cross-section of FIG. 6 illustrates one example of a lead 104 at an electrode-intersecting location, such as through a fourth electrode 208D. As shown, a distal portion of the coil conductor 502 may be coupled to an end ring member 552, which in turn is coupled (e.g., via a weld 650) to the fourth electrode 208D via a hole or slit 554 in a wall of the lead body 202. The distal portion of the coil conductor 502 may be coupled to the end ring member 552 using a variety of techniques, as further described in Zarembo, et al., U.S. Patent Application titled “INTERCONNECTIONS OF IMPLANTABLE LEAD CONDUCTORS AND ELECTRODES AND REINFORCEMENT THEREFOR,” Ser. No. 11/305,925, filed Dec. 19, 2005, which is hereby incorporated by reference in its entirety. In one example, the end ring member 552 is coupled to the conductor 502 by first urging the end ring member over a slightly larger diameter conductor. In another example, the end ring member 552 is rotary swaged to the coil conductor 502.

As the cross-sectional lead location shown in FIG. 6 is distal to a first 208A, a second 208B, and a third 208C electrode (FIG. 3), which may be coupled to one or more cable conductors 504 (FIG. 5), the distal portions of cable lumens 508, 510, 512 may need to be plugged to prevent leakage of bodily fluids, which may cause electrical shorting or corrosion. To this end, one or more thermoplastic plugs 520 may be inserted into distal portions of the cable lumens 508, 510, 512 and fused to the lead body 202 thereby sealing such lumens. In one such example, the fusable plugs 520 comprise a softer durometer than a durometer of the lead body 202 to aid the lead's flexibility and atraumaticity.

To facilitate fusing during manufacture, the materials of outer insulator 516, inner multi-lumen lead body 202, or plugs 520 may have a similar melting point temperature. The similarly between the melting point temperatures permits fusing of such insulators after softening the materials using heat (e.g., from a moving headed die, laser such as CO₂, infra-red, etc.), without a substantial disruption in their shape caused by melting. In one example, one or more of the outer insulator 516, the inner multi-lumen lead body 202, or the plugs 520 comprise one or more of PU, ETFE, PTFE, such as ePTFE, or another thermoplastic. In another example, one or more of the outer insulator 516, the inner multi-lumen lead body 202, or the plugs 520 comprise PU coated silicone rubber.

FIG. 7 is a lengthwise cross-section view of an implantable lead 104 within phantom portion 700 of FIG. 3. As shown in this example, an outer insulator 516 may be selectively disposed around a multi-lumen lead body 202 and fused 518 thereto along its full or partial length. In one example, a first lumen 506 houses a coil conductor 502 and at least a second lumen 508 houses a cable conductor 504. Advantageously, through the fusion of the outer insulator 516 to the lead body 202, a stiffness or size of the lead 104 may be tailored as desired. As shown, the outer insulator 516 is disposed around a length 702 of lead body 202, and fused along a length 704.

FIGS. 8A-8B are a lengthwise cross-sectional views of a portion of an implantable lead 104, which illustrate the lead's terminal connector section, among other things. Each lead 104 includes a lead body 202 extending from a lead proximal end portion 204 to a lead distal end portion 206 (FIG. 3), with a lead intermediate portion 302 disposed therebetween. Lead distal end portion 206 or lead intermediate portion 302 may include one or more electrodes 208A, 208B, 208C, 208D (FIG. 3) that are adapted to electrically link the lead 104 with a heart 108 (FIG. 1) or other cardiac tissue, such as vessels associated with the heart. At least one conductor 502 (coil) or 504 (cable) (FIG. 5), electrically couple electrodes 208A, 208B, 208C, 208D with lead proximal end portion 204, specifically terminal connections 304A, 304B, 304C, 304D disposed along the proximal end portion 204.

In the example of FIG. 8A, a lead terminal boot 800 comprising an outer boot 804 and an inner boot 802 is shown. One or more fusion zones 806, such as five fusion zones, bind the inner boot 802 and the outer boot 804. Each one of the fusion zones may be heated individually or all a once. In this way, the inner boot 802 and the outer boot 804 combine to form an anti-abrasive structure having a smooth, flexible, durable, and strong transition. In one example, both the inner boot 802 and the outer boot 804 comprise PU; however, the present subject matter is not so limited. Other thermoplastic polymers, such as those having different durometers or other properties, may also be used and fused together to provide optimal anti-abrasion, anti-kink, or anti-crush resistance without departing from the scope of this patent document.

In the example of FIG. 8B, a lead terminal boot 800 comprising an outer boot 804 and a two-piece inner boot 802, 803 is shown. One or more fusion zones 806, such as three fusion zones, bind a first piece of the inner boot 802 and the outer boot 804. In this example, a second piece of the inner boot 803 is held in place via entrapment by the inner boot first piece 802 and the outer boot 804 and is not fused to other portions of the lead 104. In one such example, the second piece of the inner boot 803 comprises a thermoset polymer, such as silicone rubber, while the inner boot first piece 803 and the outer boot 804 comprise a thermoplastic, such as PU. Thermoset polymers typically do not fuse well with thermoplastics (unless, for instance, the thermoset polymer is first coated with a thermoplastic polymer), and as a result, when the lead terminal boot 800 is heated, the inner boot second piece 803 does not fuse with the outer boot 804, the inner boot first piece 802, and the lead body 202. Other options for the lead terminal boot 800 include pre-molding a boot having strain relief characteristics, such accordion-like convoluted structures.

Advantageously, such lead terminal boot 800 constructions do not require the use of adhesives, rather fusion alone may provide the necessary mechanical coupling. The fusion process in many instances bonds faster than most adhesives used during lead manufacture; and thus, results in faster manufacturing output. Additionally, fusion of polymers may perform better after soak (i.e., after interaction with in vivo bodily fluids) than currently used lead adhesives found in conventional lead designs.

FIG. 9 is a lengthwise cross-section view of an implantable lead 104 (FIG. 3) within phantom portion 900 of FIG. 3, the latter of which includes a lead distal end portion 206. In this example, an atraumatic tip assembly 902 is fused at one or more fusion zones 904 to a multi-lumen lead body 202. Also shown in this example, an outer insulator 516 may be fused 518 to the lead body 202 proximal to the atraumatic tip assembly 902. In one example, the atraumatic tip assembly 902 comprises PU or other thermoplastic polymer of a softer durometer, such as Shore 80A, than the rest of the lead 104, which may comprise a durometer of Shore 55D. In another example, the atraumatic tip assembly 902 may comprise features (e.g., cut-outs or thin walls) which provide flexibility to the lead tip. The tip assembly 902 may, among other techniques, be premolded and subsequently fused to lead body 202.

Fusing atraumatic tip assembly 902 at lead distal end portion 206 provides many advantages to lead 104. As one example, the tip assembly improves maneuverability of lead 104 through tortuous vasculature, and allows for the lead to be implanted more easily and quickly than conventional leads. As another example, laser or heat fusing the tip assembly provides a seal of one or multiple lumens of lead body 202.

FIG. 10 is a lengthwise partial cutaway view illustrating a portion of an implantable lead 104. In this example, lead 104 includes a multi-lumen lead body 202 housing at least one coil conductor 502 and one cable conductor 504. In one example, lead body 202 comprises PU. Each of coil 502 and cable 504 conductors extend distally from lead proximal end portion 204 (FIG. 3), specifically from terminal connections 304A, 304B, 304C, 304D, through one or more lumens of lead body 202. In one example, the at least one coil conductor 502 comprises a polytetra-fluoroethylene (referred to as “PTFE”) tubing 1002 outside for insulation redundancy or prevention of metal ion oxidation between the metal coil and PU lead body. In another example, the at least one cable conductor 504 comprises an ETFE coating 1004. In yet another example, the at least one cable conductor 504 comprises platinum clad tantalum (referred to as “PtcladTa”). Advantageously, it is believed that PtcladTa cables don't corrode, even if exposed to the harmful in vivo environment within a subject 106 (FIG. 1).

As shown in the example of FIG. 10, a length of an outer insulator 516 is disposed over an outer diameter 514 (FIG. 5) of multi-lumen lead body 202. Surrounding outer insulator 516 is a length of heat shrink tubing 1006 having an initial diameter greater than an outer diameter of the outer insulator. Once the heat shrink tubing 1006 is positioned as desired over outer insulator 516 and lead body 202, the assembly is heated causing tubing 1006 to reduce in diametrical size. A reduction in size of heat shrink tubing 1006 imparts compressive forces on outer insulator 516 and lead body 202. The heat required to shrink tubing 1006 (e.g., low density polyethylene) further results in fusion between portions of outer insulator 516 and multi-lumen lead body 202. The assembly is subsequently allowed to cool and heat shrink tubing 1006 is removed. In one example, outer insulator 516 or the multi-lumen lead body 202 comprises PU.

Advantageously, using the heat shrink tubing 1006, an outer diameter 514 (FIG. 5) of multi-lumen lead body 202 may be reduced, as any air gaps present within the body are removed. The foregoing heat shrink technique provides the additional advantage that larger-sized conductor lumens may be made to allow for easier conductor stringing and then shrunk to a smaller size. In one example, heat shrink tubing 1006 is used to create an essentially isodiametric multi-lumen lead body 202. In another example, heat shrink tubing 1006 is selectively disposed so that some portions of lead body 202 are shrunk while other portions are not.

FIGS. 11A-11B provide lengthwise views of a (mechanical) interconnection 1100 between (portions of) a proximal lead section 1102 and (portions of) a distal lead section 1104. Specifically, FIG. 11A illustrates a lengthwise cross-sectional view of the interconnection 1100, while FIG. 11B illustrates a lengthwise exterior view of the interconnection. As shown in these examples, portions of a proximal lead section 1102 and a distal lead section 1104 may be joined together by butting a first end 1106 of the proximal lead section and a second end 1108 of the distal lead section, disposing an outer insulator 516 over the joint, disposing heat shrink tubing 1006 over outer fusable insulator 516 and the joint, and heating the assembly to get the outer insulator 516 to fuse with the multi-lumen lead bodies 202 of the proximal and distal lead sections. In varying examples, the outer insulator 516 comprises a thermoplastic having a similar melting point temperature as the lead bodies. Fusing together similar or identical materials potentially improves flex fatigue strength, because stiffness of material is similar, resulting in less of a stress concentration. Once the fusion process occurs and the interconnection 1100 cools, the heat shrink tubing 1006 may be removed.

Although not shown, additional materials may be disposed between the outer insulator 516 and the lead body 202 to potentially increase the joint strength and torque transfer characteristics of the interconnection 1100. As one example, polymer or cloth type tubular mesh or woven or braided mesh may be used. Such mesh may comprises a variety of materials, such as (but not limited to) carbon fiber, polyester fiber, expanded PTFE (referred to as “ePTFE”), long molecular chains of poly-paraphenylene terephthalamide, or metal. As another example, the strengthening material may comprise one or more fibers extending axially along the lead body 202. Optionally, identification labels 312 (FIG. 3) or fluoromarkers 310 (FIG. 3) may be embedded in one or both of the proximal or distal lead sections for monitoring purposes.

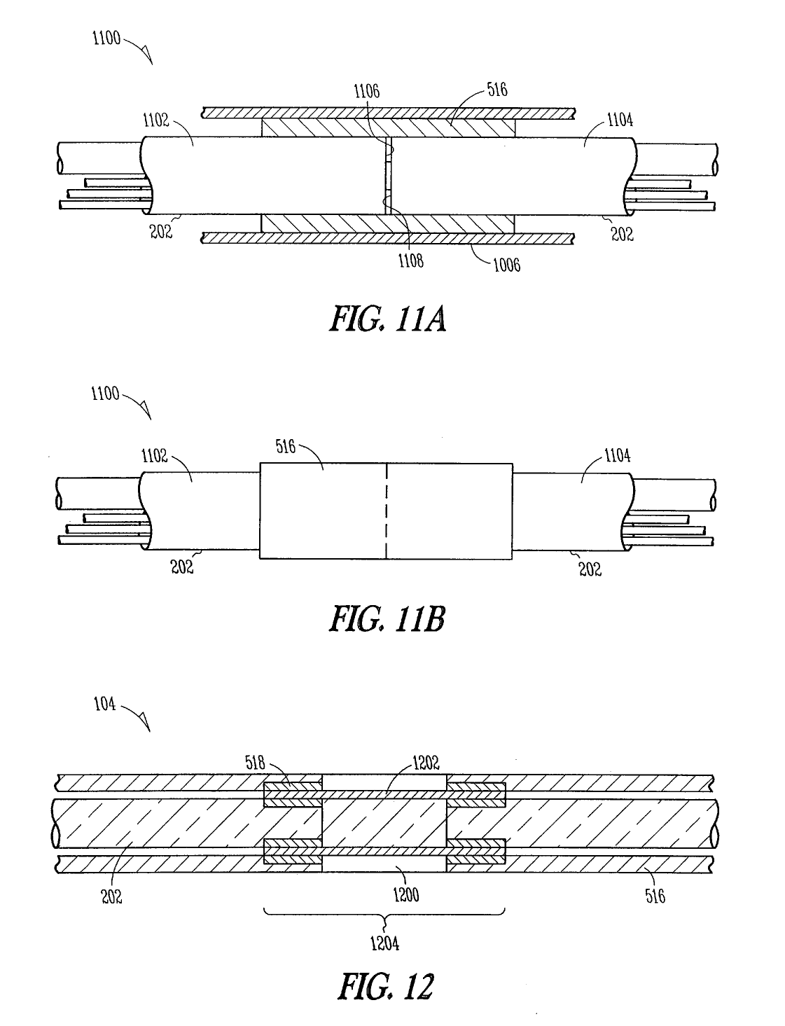

FIG. 12 is a lengthwise cross-sectional view illustrating a lead component portion 1204 of an implantable lead 104. In this example, a lead component 1200 (e.g., an electrode 208 or a drug collar 306 (FIG. 3)) is disposed on a lead body 202 and is abutted on each side by an outer insulator 516 surrounding portions of the lead body. A stiffener member 1202 is disposed between the lead body 202 and the outer insulator 516 and is fused to one or both of the same. In varying examples, the stiffener member 1202 comprises a thermoplastic tubular structure having a stiffer modulus of elasticity than a modulus of elasticity of the lead body 202 or the outer insulator 516. Through the use of the stiffener member 1202, the lead portion 1204 in the vicinity of the lead component 1200 maintains a greater overall stiffness than the adjacent portions of the lead body 202. As a result, when the lead 104 is bent, the outer insulator 516 is prevented from pulling away from the adjacent lead component 1200 edges.

Advantageously, the foregoing interconnection 1100 technique provides an adhesiveless joint that is strong and which does not result in adhesive failure concerns over time. In addition, the reduction of the outer diameter 514 (FIG. 5) of the lead bodies 202 (due to heat shrink tubing 1006) may provide room for the outer insulator 516 or any further desired (strengthening) material without increasing the pre-heat shrunk lead body size much, if at all. Furthermore, the reduction of the outer diameter 514 may allow smaller delivery catheters and introducers to be used.

The leads described herein provide numerous advantages over conventional lead designs including, among other things, one or more of: a small-sized lead body; an ability to sense or stimulate at multiple heart locations; an improved reliability in an in vivo environment; easy lead implantation and extraction; left-ventricular positioning; or varying stiffness or shape along the lead body. It is to be understood that the above description is intended to be illustrative, and not restrictive. It should be noted that the above text discusses and figures illustrate, among other things, implantable leads for use in cardiac situations; however, the present leads are not so limited. Many other embodiments and contexts, such as for non-cardiac nerve and muscle situations or for external nerve and muscle situations, will be apparent to those of skill in the art upon reviewing the above description. The scope should, therefore, be determined with reference to the appended claims, along with the full scope of legal equivalents to which such claims are entitled.

1. A lead comprising:
a multi-lumen lead body extending from a lead proximal end portion to a lead distal end portion and having a lead intermediate portion therebetween, the lead body including,
at least one heat-formed bias portion at the lead intermediate or distal end portions, and
an orientation and fixation curve portion proximal or distal to the heat-formed bias portion, the orientation and fixation curve including one or more heat-formed polymers;
at least three tissue sensing/stimulation electrodes disposed along the lead intermediate or distal end portions;
one or more terminal connections disposed along the lead proximal end portion;
one or more conductors contained within the multi-lumen lead body extending between the at least three tissue sensing/stimulation electrodes and the one or more terminal connections.

2. The lead as recited in claim 1, wherein the lead body comprises an environment adaptable polymer, whereby one or both the at least one heat-formed bias portion of the orientation and fixation curve adapts over time at a temperature less than or equal to body temperature to a coronary vasculature geometry in which it is implanted.

3. The lead as recited in claim 1, wherein the heat-formed bias portion comprises at least one of a cylindrical, oval, or cam helical shape.

4. The lead as recited in claim 3, wherein the cylindrical, oval, or cam helical shape extends along a preformed longitudinal curve having of an axis of the lead body, the preformed longitudinal curve having a radius substantial similar to a radius of a great cardiac vein.

5. The lead as recited in claim 3, wherein the at least three tissue sensing/ stimulation electrodes are spaced about 90 degrees apart from one another as measured from an axis of the helical shape.

6. The lead as recited in claim 1, wherein the heat-formed bias portion comprises at least one of a sinusoidal curve or J-shape.

7. The lead as recited in claim 1, wherein the heat-formed bias portion comprises an elasticity that is substantially the same as one or more portions of a heart.

8. The lead as recited in claim 1, further comprising a flexible tip disposed at the lead distal end portion, the flexible tip being more flexible than the lead body and fused to the lead body at one or more fusion zones.

9. The lead as recited in claim 8, wherein the flexible tip comprises at least one of a thermoplastic polymer or a thermoplastic-coated thermoset polymer.

10. (canceled)

11. A lead comprising:
a thermoplastic lead body extending from a lead proximal end portion to a lead distal end portion and having a lead intermediate portion therebetween, the lead body including one or more longitudinally extending lumens;
a first conductor received in, and extending along, a first lumen;
one or more tissue sensing/stimulation electrodes disposed along the lead intermediate or distal end portions and coupled with the first conductor;
a thermoplastic outer insulator disposed around a portion of the lead body; and
a fusion zone disposed between the thermoplastic lead body and the thermoplastic outer insulator, the fusion zone including a union of the lead body and the outer insulator.

12. The lead as recited in claim 11, wherein the lead body comprises at least one of a thermoplastic polymer or a thermoplastic-coated thermoset polymer.

13. The lead as recited in claim 11, further comprising one or more lead body fusable plugs disposed adjacent a distal end of the first or second conductors.

14. The lead as recited in claim 13, wherein the one or more lead body fusable plugs comprise one or more thermoplastics having a first durometer which is less than a second durometer of the lead body.

15. The lead as recited in claim 11, wherein the lead body and the outer insulator comprise materials having a substantially similar melting temperature.

16. The lead as recited in claim 11, wherein the outer insulator is disposed around, and fused to, a portion of the lead body adjacent a lead component.

17. The lead as recited in claim 11, further comprising a fixation member disposed at the lead intermediate or the lead distal end portions, the fixation member fused to the lead body.

18. A lead comprising:
a lead body having one or more longitudinally extending lumens therein;
at least one conductor surrounded by a polymer coating, the at least one conductor received in, and extending along, the one or more lumens;
an outer insulator surrounding a portion of the lead body;
a length of heat shrink tubing disposed around the outer insulator, the heat shrink tubing having a non-shrunk inner diameter greater than an initial outer diameter of the outer insulator; and
wherein the heat shrink tubing diametrically compresses the outer insulator and the lead body when heated, such heat fusing portions of the outer insulator with the lead body.

19. The lead as recited in claim 18, wherein the at least one conductor comprises a coil conductor and the polymer coating surrounding the coil conductor comprises polytetrafluoroethylene.

20. The lead as recited in claim 18, wherein the at least one conductor comprises a cable conductor and the polymer coating surrounding the cable conductor comprises one or more of ethylene tetrafluoroethylene, polytetrafluoroethylene, or expanded polytetrafluoroethylene.

21. The lead as recited in claim 18, wherein one or both of the lead body or the outer insulator comprise polyurethane.

22. A lead comprising:
a lead body adapted to carry signals, the lead body extending from a lead proximal end portion to a lead distal end portion, and having a lead intermediate portion therebetween;
a connector assembly located at the lead proximal end portion;
at least one conductor disposed within the lead body, the at least one conductor electrically coupling the connector assembly to one or more tissue sensing/stimulation electrodes;
a lead terminal boot including an inner boot and an outer boot, the inner boot fusable with the lead body on an inner boot inner surface and fusable with the outer boot on an inner boot outer surface; and
wherein the lead terminal boot is disposed on the lead body distal to the connector assembly.

23. The lead as recited in claim 22, wherein a portion of one or both of the inner boot or the outer boot comprises at least one of a thermoplastic polymer or a thermoplastic-coated thermoset polymer.

24. The lead as recited in claim 22, wherein one or both of the inner boot or the outer boot comprises a thermoplastic polymer having a first durometer which is different than a second durometer of the lead body.

25. The lead as recited in claim 22, wherein one or both of the inner boot or the outer boot includes at least one strain-relief structure.

26. The lead as recited in claim 22, wherein the inner boot is fused to the lead body at an inner boot proximal and distal end; and wherein the outer boot is fused to the inner boot at an outer boot distal end and fused to the connector assembly at an outer boot proximal end.

27. A lead assembly comprising:
a proximal lead section and a distal lead section, an end portion of the proximal lead section disposed adjacent to an end portion of the distal lead section;
an outer insulator disposed around an outer surface of the proximal lead section end portion and the distal lead section end portion;
a length of heat shrink tubing disposed around the outer insulator, the length of the heat shrink tubing substantially covering the outer insulator; and
wherein the heat shrink tubing diametrically compresses the outer insulator, a lead body of the proximal lead section, and a lead body of the distal lead section when heated, such heat fusing portions of the outer insulator with the lead body of the proximal lead section and lead body of the distal lead section.

28. The lead assembly as recited in claim 27, further comprising a strengthening material disposed between the outer insulator and the lead body.

29. The lead assembly as recited in claim 28, wherein the strengthening material comprises a tubular mesh or braided structure including one or more of carbon fiber, polyester fiber, expanded polytetrafluoroethylene, poly-paraphenylene terephthalamide, or metal.

30. The lead assembly as recited in claim 28, wherein the strengthening material comprises one or more fibers extending axially along the lead body.

31. A lead comprising:
a lead body having one or more longitudinally extending lumens therein;
at least one conductor received in, and extending along, the one or more lumens;
a lead component disposed on the lead body;
an outer insulator surrounding a portion of the lead body;
a stiffener member having a length greater than a length of the lead component such that one or more stiffener member portions extend proximal and distal to the lead component, the stiffener member disposed between the lead body and the outer insulator; and
wherein the one or more proximal and distal extending portions of the stiffener member are fused to one or both of the lead body or the outer insulator.

32. The lead as recited in claim 31, wherein an outer diameter of the lead component is substantially the same as an outer diameter of the outer insulator.

33. The lead as recited in claim 31, wherein the lead component comprises an electrode which is electrically coupled to the at least one conductor.

34. The lead as recited in claim 31, wherein the stiffener member comprises a thermoplastic tubular structure having a first modulus of elasticity which is stiffer than a second modulus of elasticity of the lead body and a third modulus of elasticity of the outer insulator.

35. The lead as recited in claim 2, wherein the environmental adaptable polymer is selected based on its glass transition temperature.
